(19) United States
(12) Patent Application Publication
(10) Pub. No.: US 2008/0154098 A1
(43) Pub. Date: Jun. 26, 2008

(54) Apparatus for monitoring physiological, activity, and environmental data

(76) Inventors: Margaret Morris, Portland, OR (US); Terry Dishongh, Portland, OR (US); Farzin Guilak, Beaverton, OR (US)

Correspondence Address: Intel Corporation;c/o DARBY & DARBY P.C., P.O. BOX 770, CHURCH STREET STATION, NEW YORK, NY, 10008-0770, US

(21) Appl. No.: 11/641,973

(22) Filed: Dec. 20, 2006

(51) Int. Cl. A61B 5/00

(52) U.S. Cl. 600/300

(57) ABSTRACT

The invention relates to an earpiece form factor including technology to monitor physiological, activity and environmental data on a user. The device includes a pulse oximeter unit to provide blood oxygenation level and beat-to-beat timing, a three-axis accelerometer to provide orientation and activity level, and a temperature sensor to provide a subject's skin temperature. The device may also capture other forms of data for the user and the user's surroundings. Captured data are transmitted wirelessly to a mobile phone, PDA or other device that supports wireless transmission, and enables monitoring form another location.

### FIELD OF INVENTION

The invention relates to an apparatus for monitoring physiological, activity and environmental data.

### BACKGROUND

Pulse Oximetry was developed by Nellcor Incorporated in 1982, and introduced into the US operating room market in 1983. Prior to its introduction, a patient's oxygenation was determined by a painful arterial blood gas, a single point measure which typically took a minimum of 20-30 minutes processing by a laboratory. (In the absence of oxygenation, damage to the brain starts in 5 minutes with brain death in another 10-15 minutes). In the US alone, approximately $2 billion was spent annually on this measurement. With the introduction of pulse oximetry, a non-invasive, continuous measure of patient's oxygenation was possible, revolutionizing the practice of anesthesia and greatly improving patient safety. Prior to its introduction, studies in anesthesia journals estimated US patient mortality as a consequence of undetected hypoxemia at 2,000 to 10,000 deaths per year, with no known estimate of patient morbidity.

By 1987, the standard of care for the administration of a general anesthetic in the US included pulse oximetry. From the operating room, the use of pulse oximetry rapidly spread throughout the hospital, first in the recovery room, and then into the various intensive care units. Pulse oximetry was of particular value in the neonatal unit where the patients do not thrive with inadequate oxygenation, but also can be blinded with too much oxygen. Furthermore, obtaining an arterial blood gas from a neonatal patient is extremely difficult.

In 2005 Masimo Corporation introduced the first FDA-approved pulse oximeter to monitor carbon monoxide levels non-invasively. Several products currently exist that enable patients to monitor their exertion. For example, Nonin™ has a Bluetooth enabled pulse oximeter, that straps to the subject's wrist and has a clip on one fingertip connected to the main unit with a cable. The cumbersome form factor of this unit precludes its use during daily activities that require unencumbered availability of the hands or fingers (for example, it would be very difficult to type while wearing the Nonin oximeter). Also, the lack of activity or temperature sensors on the Nonin limits the range of applications. The Army Research Laboratory has developed a sensor to monitor physiologic and motor activities acoustically. The sensor consists of a hydrophone (piezo transducer) in a gel-filled small rubber pad. This sensor enables high SNR capture of cardiac, respiratory, voice, and other data. It is worn using a harness and can be placed on the torso, neck, or head.

However, these products are limited in their use and cumbersome. These factors preclude use during daily activities that require, for example, unencumbered availability of the hands or fingers (which would prevent, for example, typing) and the range of applications is limited.

### BRIEF DESCRIPTION OF THE DRAWINGS

The invention is described below in more detail with reference to the exemplary embodiments and drawings, in which:

FIG. 1. illustrates an exemplary high-level architecture in accordance with the invention.

FIG. 2. illustrates an exemplary earpiece circuit board in accordance with the invention.

FIG. 3. illustrates the exemplary earpiece of FIG. 2. with a custom enclosure.

### DETAILED DESCRIPTION

A pulse oximeter is a medical device that indirectly measures the amount of oxygen in a patient's blood and changes in blood volume in the skin, a photoplethysmograph. It is often attached to a medical monitor so staff can see a patient's oxygenation at all times. Most monitors also display the heart rate.

A blood-oxygen monitor displays the percentage of arterial hemoglobin in the oxyhemoglobin configuration. Acceptable normal ranges are from 95 to 100 percent. For a patient breathing room air, at not far above sea level, an estimate of arterial pO₂ can be made from the blood-oxygen monitor SpO₂ reading.

A pulse oximeter is a particularly convenient non-invasive measurement instrument. Typically it has a pair of small light-emitting diodes facing a photodiode through a translucent part of the patient's body, usually a fingertip or an earlobe. One LED is red, with wavelength of 660 nm, and the other is infrared, 910 nm. Absorption at these wavelengths differs significantly between oxyhemoglobin and its deoxygenated form, therefore from the ratio of the absorption of the red and infrared light the oxy/deoxyhemoglobin ratio can be calculated.

The monitored signal bounces in time with the heart beat because the arterial blood vessels expand and contract with each heartbeat. By examining only the varying part of the absorption spectrum (essentially, subtracting minimum absorption from peak absorption), a monitor can ignore other tissues or nail polish and discern only the absorption caused by arterial blood. Thus, detecting a pulse is essential to the operation of a pulse oximeter and it will not function if there is none.

Because of their simplicity and speed (they clip onto a finger and display results within a few seconds), pulse oximeters are of critical importance in emergency medicine and are also very useful for patients with respiratory or cardiac problems, as well as pilots operating in a non-pressurized aircraft above 10,000 feet (12,500 feet in the US), where supplemental oxygen is required. Prior to the oximeter's invention, many complicated blood tests needed to be performed.

The pulse oximeters could use digital signal processing to make accurate measurements in clinical conditions that were otherwise impossible. These could include situations of patient motion, low perfusion, bright ambient light, and electrical interference. Because of their insensitivity to non-pulsate signals, it is also possible to build reflectance probes that place the photodiode beside the LEDs and can be placed on any flat tissue. These can be used on non-translucent body parts, to measure pulses in specific body parts (useful in plastic surgery), or when more convenient sites are unavailable (severe burn victims). They could be applied to the forehead of patients with poor peripheral perfusion.

Oximetry is not a complete measure of respiratory sufficiency. A patient suffering from hypoventilation (poor gas exchange in the lungs) given 100% oxygen can have excellent blood oxygen levels while still suffering from respiratory acidosis due to excessive carbon dioxide.

Nor is it a complete measure of circulatory sufficiency. If there is insufficient bloodflow or insufficient hemoglobin in the blood (anemia), tissues can suffer hypoxia despite high oxygen saturation in the blood that does arrive.

It also should be noted that two-wavelength saturation level measurement devices can not distinguish carboxyhemoglobin due to carbon monoxide inhalation from oxyhemoblobin, which must be taken into account when diagnosing a patient in emergency rescue from, e.g., a fire in an apartment. A CO-oximeter measures absorption at additional wavelengths to distinguish CO from O₂ and determine the blood oxygen saturation more reliably.

Heart failure and other cardiac patients need to monitor their exertion. Exercise and other stimulation offer important benefit but are often avoided because they pose serious risks. Real time feedback would allow people to monitor and modulate exertion, and the ability to safely and confidently pursue activities with preventive value.

The embodiments of the invention include devices that include consciousness monitors for anesthesia/sedation, which works by using a sensor that is placed on the patient's forehead to measure electrical activity in the brain and translate it into a number between 100 (wide awake) and zero (absence of brain electrical activity). Another such example consists of an armband that monitors caloric expenditure and communicates to a web site for users/trainers. Wireless transceivers can communicate with a variety of third-party monitors. Transceiver data is sent to a wireless gateway or armband, and from there to a call center via Internet.

A sensor device worn on a user to monitor physiological, activity and environmental data of the user, comprising an oximeter unit to measure oxygenation level and heart rate; an accelerometer to measure activity level; and a temperature sensor to measure a temperature level. The sensor device communicates with the mobile device using Bluetooth technology. The sensor device is one of an earpiece, hearing aid or telephone headset. The sensor device communicates to the phone via a Body Area Network (BAN)—a short-range wireless network to transmit monitored data. The cell phone securely transmits the data is through the Internet over a Wide Area Network (WAN) for storage on a back-end server. From there it can be accessed by home users, researchers, clinicians, etc. through an authenticated, secure connection.

A method of monitoring physiological, activity, and environmental data of a user wearing a sensor device, comprising measuring oxygenation level and heart rate with an oximeter unit; measuring activity level with an accelerometer; and measuring a temperature level with a temperature sensor. The sensor device communicates with the mobile device using Bluetooth technology. The sensor device is one of an earpiece, hearing aid or telephone headset. The sensor device communicates via a network to transmit monitored data. The transmitted data is sent to one of a home computer, researcher workstation and storage server.

A system to transmit data acquired by a sensor worn by a user, comprising a sensor to acquire using at least one of an oximeter unit, accelerometer and temperature sensor located in the sensor; a mobile device receiving the data from the sensor of the user; and a network to receive the data transmitted from the mobile device to at least one of a home server, workstation and storage server. The acquired data is sent from the sensor to the mobile device using Bluetooth technology. The sensor is an earpiece worn by the user. The data transmitted over the network is monitored at least one of the home server, workstation and storage server.

A method of transmitting data acquired by a sensor worn by a user over a network, comprising acquiring data from the sensor using at least one of an oximeter unit, accelerometer and temperature sensor located in the sensor; sending the data from the sensor to a mobile device of the user; and transmitting the data sent to the mobile device over the network. The network comprises a home server, a workstation and a storage server. The acquired data is sent from the sensor to the mobile device using Bluetooth technology. The sensor is an earpiece worn by the user. The data transmitted over the network is monitored at least one of the home server, workstation and storage server.

The invention includes an earpiece form factor, similar to a “behind-the-ear” hearing aid or Bluetooth™ telephone headset, including technologies to monitor physiological, activity, and environmental data on a user. The invention, in one embodiment, includes a pulse oximeter unit (providing blood oxygenation level and heart rate), a three-axis accelerometer (providing activity level), and temperature sensor (providing subject's skin temperature). Other embodiments include technologies to capture other types of data from the body and its surroundings. Captured data are transmitted wirelessly to a cell phone, PDA, or other device that supports wireless radio and protocol implemented in the earpiece (for example, Bluetooth, Zigbee, or a proprietary system). This unit serves to process data and transmit it to servers for storage and/or further processing. It also provides for feedback and actuation to the user.

In another embodiment of the present invention, pulse oximetry is unplugged and embedded into a wireless earpiece of a mobile phone. Immediate feedback appears on the phone screen regarding exertion levels (including warnings to slow down) or a longitudinal view which plots cardiovascular stress against activity level.

Currently, there is no technology that allows monitoring physiological, activity, and environmental data in a small, wearable, wireless form factor. The combination of these parameters allows for functionality not possible with current units that typically monitor a single parameter.

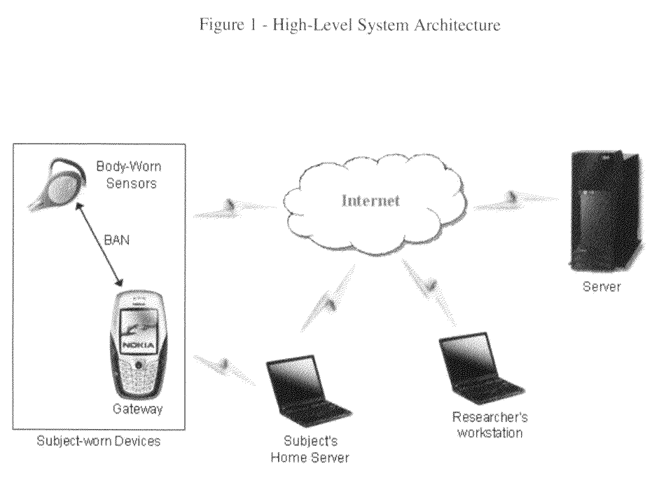

FIG. 1 illustrates a high-level architecture of an embodiment in the invention. The depicted architecture includes subject-worn devices, such as an earpiece and mobile phone, a home server, a researcher workstation, a clinician workstation, and a server, each of which may be connected via a network, such as the Internet. As the description below indicates, the earpiece is capable of monitoring the user to record physiological, activity and environmental information, and transmit or send such information to a mobile device worn by the user, for example via Bluetooth technology. This information may be displayed on the user mobile device (e.g. cell phone) and/or transmitted via the network to a user's home computer, a researcher workstation (such as a doctor's office) or a server for storage.

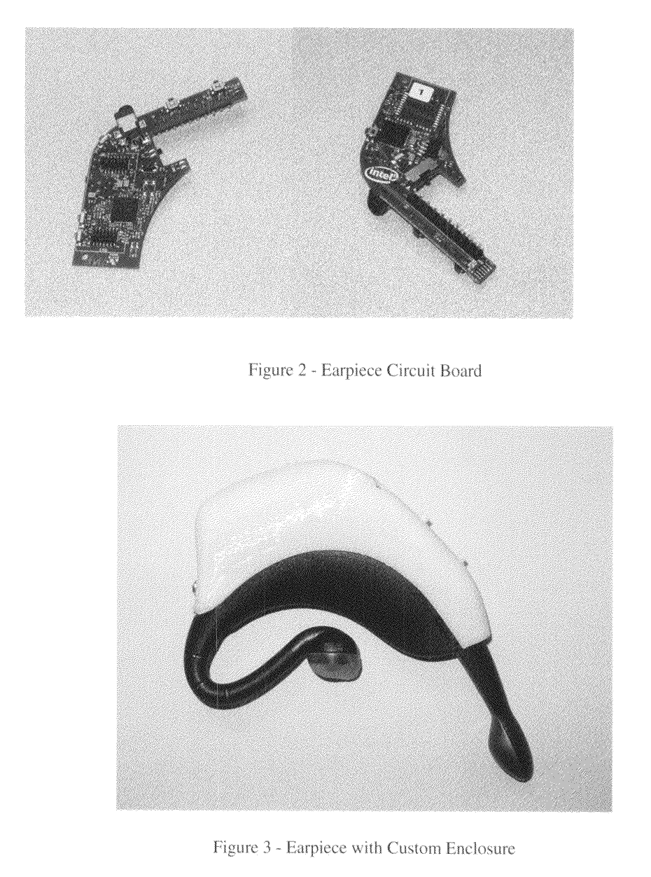

FIGS. 2 and 3 illustrate an earpiece circuit board and the custom enclosure of the earpiece, respectively. These illustrations are exemplary of the type of device that may be worn by the user.

In one embodiment, this device allows monitoring the subject's stress level: an increase in heart rate that is not preceded by activity (as measured by the accelerometer) is likely to be caused by increased stress. Similarly, an increase in skin temperature following activity may be a normal physiological response, but without activity may indicate illness. Features present on the cell phone device allow for audio, visual, and tactile feedback to the subject. The presence of a microphone enables capturing voice signals for analysis: there is research indicating that early detection and progression of certain neurological diseases is possible through voice analysis. The presence of an earpiece allows for audio cueing and prompting to be provided to the subject. Audio cues are an active area of research in falls, which may assist prevention for in patients with certain neurological conditions. Audio reminders may be an effective means to remind subjects to perform necessary activities (like taking medication). Also, the compact form factor will allow researchers to gather data that may difficult to obtain otherwise. The earpiece is worn primarily for phone communication but also affords continuous, ecologically sensitive health monitoring. The earpiece is not stigmatizing and may appeal to people who are concerned about health and wellness, but do not want to announce these concerns publicly.

The invention provides integration of physiological, activity, and environmental monitors in a commonly-used form factor, which also is capable of providing real-time feedback to the wearer. This is particularly true because the point of sensor fusion and feedback is a single integrated unit. Essentially, the invention provides integration of the devices into a commonly used wearable platform to obtain pulse oximetry, heart rate, and temperature from one device to monitor for conditions such as: medication adherence, glycemic shock, and heart rate variability. The device may also combine sensors capable of tracking head motion with blood oxygen level as a leading indicator for preventing the occurrence of falls. Additionally, the device may provide on-body feedback in response to monitored physiological signals. These can be used to affect changes in wearer's behavior to benefit their health. An ear-worn device with motion sensors can also be used as a pedometer. Combining it with heart rate gives an indication of the effectiveness of physical activity. Adding a cell phone gives the ability to do location tracking to map measured distance traveled to caloric expenditure. The cell phone also allows us to capture, store, forward, and analyze data from the wearer.

Physiological: Human physiology is the science of the mechanical, physical, and biochemical functions of normal humans or human tissues or organs. The principal level of focus of physiology is at the level of organs and systems. Most aspects of human physiology are closely homologous to corresponding aspects of animal physiology, and animal experimentation has provided much of the foundation of physiological knowledge. Human physiology is one of the basic sciences of medical study, and as such is most often applied as medical care.

Environmental: Environmental physiology is a biological discipline which studies the adaptation of organism's physiology to environmental conditions.

Oximetry: Pulse oximetry is a non-invasive method which allows health care providers to monitor the oxygenation of a patient's blood. A sensor is placed on a relatively thin part of the patient's anatomy, usually a fingertip or earlobe, or in the case of a neonate, across a foot, and red and infrared light is passed from one side to the other. Changing absorbance of each of the two wavelengths is measured, allowing determination of the absorbances due to the pulsing arterial blood alone, factoring out venous blood, skin, bone, muscle, fat, and even fingernail polish. Based upon the ratio of changing absorbances of the red and infrared light caused by the difference in color between oxygen-bound (bright red) and unbound (dark red or in severe cases blue) hemoglobin in the blood, a measure of oxygenation (the percent of hemoglobin molecules bound with oxygen molecules) can be made.

Accelerometer: An accelerometer is a device for measuring acceleration. An accelerometer inherently measures its own motion (locomotion), in contrast to a device based on remote sensing. One application for accelerometers is to measure gravity, wherein an accelerometer is specifically configured for use in gravimetry. Such a device is called a gravimeter. Accelerometers are used along with gyroscopes in inertial guidance systems, as well as in many other scientific and engineering systems. One of the most common uses for micro electro-mechanical system (MEMS) accelerometers is in airbag deployment systems for modern automobiles. In this case the accelerometers are used to detect the rapid negative acceleration of the vehicle to determine when a collision has occurred and the severity of the collision. An accelerometer is an instrument for measuring acceleration, detecting and measuring vibrations, or for measuring acceleration due to gravity (inclination). Accelerometers can be used to measure vibration on vehicles, machines, buildings, process control systems and safety installations. They can also be used to measure seismic activity, inclination, machine vibration, dynamic distance and speed with or without the influence of gravity. Accelerometers are perhaps the simplest MEMS device possible, sometimes consisting of little more than a suspended cantilever beam or proof mass (also known as seismic mass) with some type of deflection sensing and circuitry. MEMS Accelerometers are available in a wide variety of ranges up to thousands of gₙ's. Single axis, dual axis, and three axis models are available.

It is readily understood by the skilled artisan that the embodiments disclosed herein are merely exemplary and are not intended to limit the scope of the invention.

1. A sensor device worn on a user to monitor physiological, activity and environmental data of the user, comprising:
an oximeter unit to measure oxygenation level and/or beat-to-beat timing;
an accelerometer to detect orientation and to measure activity level;
and a temperature sensor to measure a temperature level
wherein the oximeter unit, the accelerometer and the temperature sensor are integrally combined within a single unit of the sensor device so as to allow simultaneous functionality of the oximeter unit, the three-axis accelerometer and the temperature sensor.

2. The sensor device according to claim 1, wherein the sensor device communicates wirelessly with a mobile device.

3. The sensor device according to claim 2, wherein the sensor device communicates wirelessly with the mobile device.

4. The sensor device according to claim 1, wherein the sensor device is one of an earpiece, hearing aid or telephone headset.

5. The sensor device according to claim 1, wherein the sensor device communicates via a network to transmit monitored data.

6. The sensor device according to claim 5, wherein the transmitted data is sent to one of a home computer, researcher workstation, clinician workstation, and storage server.

7. A method of monitoring physiological, activity and environmental data of a user wearing a sensor device, comprising:
measuring oxygenation level and/or beat-to-beat timing with an oximeter unit;
measuring activity level with an accelerometer; and
measuring a temperature level with a temperature sensor
wherein the oximeter unit the accelerometer and the temperature sensor are integrally combined within a single unit of the sensor device so as to allow simultaneous functionality of the oximeter unit, the three-axis accelerometer and the temperature sensor.

8. The method according to claim 1, wherein the sensor device communicates wirelessly with a mobile device.

9. The method according to claim 8, wherein the sensor device communicates wirelessly with the mobile device.

10. The method according to claim 7, wherein the sensor device is one of an earpiece, hearing aid or telephone headset.

11. The method according to claim 7, wherein the sensor device communicates via a network to transmit monitored data.

12. The method according to claim 11, wherein the transmitted data is sent to one of a home computer, clinician workstation, researcher workstation and storage server.

13. A system to transmit data acquired by a sensor worn by a user, comprising:
a sensor to acquire using at least one of an oximeter unit, accelerometer and temperature sensor located in the sensor;
a mobile device receiving the data from the sensor of the user; and
a network to receive the data transmitted from the mobile device to at least one of a home server, clinician workstation, researcher workstation and storage server
wherein the oximeter unit, the accelerometer and the temperature sensor are integrally combined within a single unit of the sensor device so as to allow simultaneous functionality of the oximeter unit, the three-axis accelerometer and the temperature sensor.

14. The system of claim 13, wherein the acquired data is sent wirelessly from the sensor to the mobile device.

15. The system of claim 13, wherein the sensor is an earpiece worn by the user.

16. The system of claim 13, wherein data transmitted over the network is monitored by at least one of the home server, clinician workstation, researcher workstation and storage server.

17. A method of transmitting data acquired by a sensor worn by a user over a wireless network, comprising:
acquiring data from the sensor using at least one of an oximeter unit, accelerometer and temperature sensor located in the sensor;
sending the data from the sensor to a mobile device of the user; and
transmitting the data sent to the mobile device over the network
wherein the oximeter unit, the accelerometer and the temperature sensor are integrally combined within a single unit of the sensor device so as to allow simultaneous functionality of the oximeter unit, the three-axis accelerometer and the temperature sensor.

18. The method of claim 17, wherein the network comprises a home server, a workstation and a storage server.

19. The method of claim 17, wherein the acquired data is sent from the sensor to the mobile device.

20. The method of claim 17, wherein the sensor is an earpiece worn by the user.

21. The method of claim 18, wherein data transmitted over the network is monitored at least one of the home server, workstation and storage server.

22. The sensor device of claim 1, wherein the device has a wearable, wireless form factor.
